(19) United States
(12) Patent Application Publication
(10) Pub. No.: US 2021/0391048 A1
(43) Pub. Date: Dec. 16, 2021

(54) CARE PLAN ASSIGNMENT BASED ON CLUSTERING

(71) Applicant: KONINKLIJKE PHILIPS N.V., EINDHOVEN (NL)

(72) Inventors: Jan Johannes Gerardus DE VRIES, Leende (NL); Joep Joseph Benjamin Nathan VAN BERKEL, Gronsveld (NL); Ioanna SOKORELI, Eindhoven (NL)

(21) Appl. No.: 17/288,220

(22) PCT Filed: Apr. 30, 2020
(86) PCT No.: PCT/EP2019/078701

§ 371 Date: Apr. 23, 2021

Related U.S. Application Data
(60) Provisional application No. 62/749,699, filed on Oct. 24, 2018.

(51) Int. Cl. G16H 20/00; G16H 10/60

(52) U.S. Cl. CPC G16H 20/00; G16H 10/60

(57) ABSTRACT

A method for assigning a care plan to a patient, the method including: clustering patients based upon input patient data; producing a care plan frequency distribution for each cluster based upon the care plans assigned to each patient in the cluster; and assigning, for each cluster, the most frequent care plan from the frequency distribution for each cluster to each patient in that cluster.

### TECHNICAL FIELD

Various exemplary embodiments disclosed herein relate generally to care plan assignment based on clustering

### BACKGROUND

Population Health Management (PHM) tries to improve clinical and financial outcomes on an aggregated population level by grouping patients with similar characteristics, through monitoring and improving care delivered to individual patients within a group.

This requires aggregation of patient data across multiple health information data sources along the care continuum, the analysis of this data, the optimal grouping of patients with similar characteristics and supporting care providers to improve clinical and financial outcomes by optimizing patient care plans.

Health care providers do not typically use the same electronic medical record (EMR) systems, so the aggregation of data and effective communication can be difficult. Even when healthcare providers do use the same clinical systems, it can be tedious to reconstruct a patient's longitudinal record, let alone study the similarities between multiple patients' records. Furthermore, relevant information pertaining to a patient or populations' health goes beyond an individual's individual patient record. Additional information such as insurance claims or socio-economic factors are crucial to understand the health context. This missing link between the different data sources might cause gaps in care or make it difficult to derive knowledge and insights about the population of patients cared for. Recently, companies have started to offer population health management software that aggregate data from the EMR, claims systems or other sources, and connect hospitals, providers, physicians, care managers and beneficiaries, providing the means to improve the value of care and optimizing delivery of care and containing costs.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for assigning a care plan to a patient, the method including: clustering patients based upon input patient data; producing a care plan frequency distribution for each cluster based upon the care plans assigned to each patient in the cluster; and assigning, for each cluster, the most frequent care plan from the frequency distribution for that cluster to each patient in that cluster.

Various embodiments are described, further including determining the homogeneity of each cluster; determining if the homogeneity of any cluster is less than a first threshold; and re-clustering any clusters that have a homogeneity that is less than the first threshold.

Various embodiments are described, further including determining the homogeneity of each cluster; determining if the homogeneity of any cluster is less than a first threshold; and discarding any clusters that have a homogeneity that is less than the first threshold.

Various embodiments are described, further including determining if the frequency of no care plan of any cluster is greater than a second threshold; and discarding any cluster with a frequency of no care plan greater than the second threshold.

Various embodiments are described, further including determining if the frequency of no care plan of any cluster is greater than a second threshold; and producing an alert to a user indicating the cluster with a frequency of no care plan greater than the second threshold.

Various embodiments are described, further including producing a confidence measure for each patient whose care plan assignment changed when assigning, for each cluster, the most frequent care plan from the frequency distribution for that cluster to each patient in that cluster.

Various embodiments are described wherein producing a confidence measure further comprises determining a distance between each patient whose care plan assignment changed and those patients in the cluster that were already assigned to the most frequent care plan from the frequency distribution.

Various embodiments are described, further including presenting the assigned care plan for a specific patient to a user; receiving input from the user to modify the care plan assignment of the specific patient based upon patient constraints; and modifying the care plan assignment of the specific patient based upon the user input.

Various embodiments are described, further including presenting the assigned care plans for the patients to a user; receiving input from the user to approve the care plan assignments of the patients; and initiating enrollment of patients in the assigned care plan for patients whose care plan assignment has changed.

Further various embodiments relate to a method for assigning a care plan to a patient, the method including: clustering patients based upon input patient data; determining for each cluster which care plan assigned to patients in that cluster provides the best outcome; and assigning, for each cluster, the care plan that provides the best outcome for that cluster to each patient in that cluster.

Various embodiments are described, wherein the best outcome is one of the best success rate of the care plan or the lowest cost successful care plan.

Various embodiments are described, further including determining the homogeneity of each cluster; determining if the homogeneity of any cluster is less than a first threshold; and re-clustering any clusters that have a homogeneity that is less than the first threshold.

Various embodiments are described, further including determining the homogeneity of each cluster; determining if the homogeneity of any cluster is less than a first threshold; and discarding any clusters that have a homogeneity that is less than the first threshold.

Various embodiments are described, further including producing a confidence measure for each patient whose care plan changed when assigning, for each cluster, the care plan that provides the best outcome for each cluster to each patient in that cluster.

Various embodiments are described, wherein producing a confidence measure further comprises determining a distance between each patient whose care plan assignment changed and those patients in the cluster that were already assigned to the care plan that provides the best outcome.

Various embodiments are described, further including presenting the assigned care plan for a specific patient to a user; receiving input from the user to modify the care plan assignment of the specific patient based upon patient constraints; and modifying the care plan assignment of the specific patient based upon the user input.

Various embodiments are described, further including presenting the assigned care plans for the patients to a user; receiving input from the user to approve the care plan assignments of the patients; and initiating enrollment of patients in the assigned care plan for patients whose care plan assignment has changed.

### BRIEF DESCRIPTION OF THE DRAWINGS

In order to better understand various exemplary embodiments, reference is made to the accompanying drawings, wherein:

FIG. 1 illustrates a flow diagram illustrating a method of assigning care plans to patients; and

FIG. 2 illustrates a histogram of the care plans in which the patients in the cluster are enrolled.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

A “gap in care” is defined as the discrepancy between recommended best practices and the care that is actually provided. Closing gaps in care is essential to improve health outcomes, increase patient satisfaction, and reduce cost in health care. Currently care professionals only receive and extract very limited information on which care gaps to address and recommendations on how to address these gaps.

Reasons why they lack insight on these gaps are the lack of or limited availability of or access to data, and the limited time to process this information. Usually, when care providers/care managers want to assign a care program to a patient, they only have a partial view of the patient's health status and might be missing out valuable health status related characteristics of the patient; furthermore, they might be excluding other patients that would fit this program because they are not in the care professionals' scope (list of patients to address) for this particular condition.

An embodiment of a method will now be described that uses as input data from the EMR system and potentially other sources (e.g., claims-based systems, lab-systems, socio-economic sources, etc.) where each patient is characterized in terms of at least one of the following data types: clinical, medical, claims, demographic, socioeconomic, utilization.

The method may include the following set of steps. First the patients are clustered to obtain groups of similar patients based upon the input data. Then for each cluster a measure of homogeneity is determined, an inventory is made of which care plans are assigned to the patients in that cluster, a modified care-plan-assignment is suggested to the user, and the user can make changes and/or acknowledge the assignment. Each of these steps will now be described.

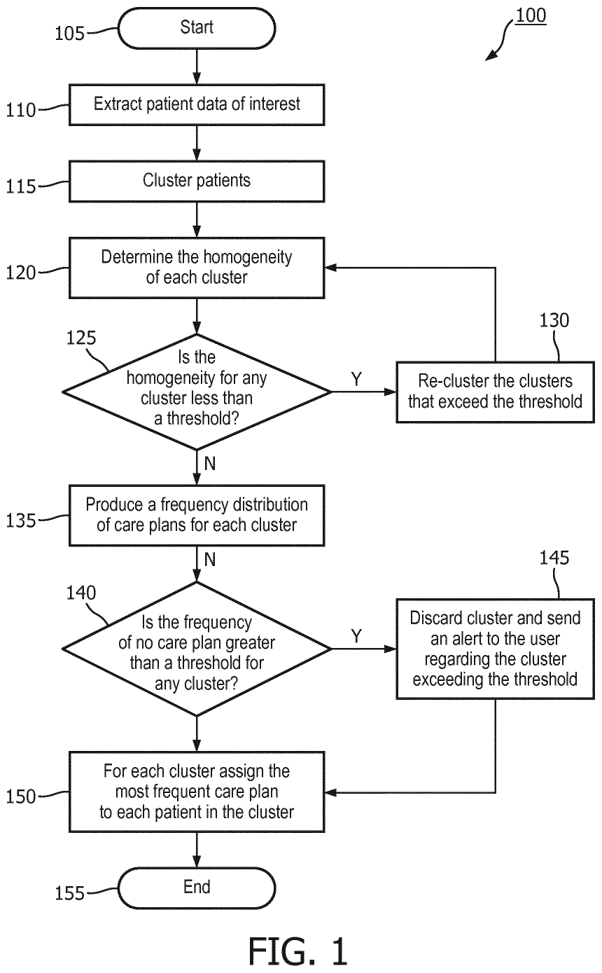

FIG. 1 illustrates a flow diagram illustrating a method 100 of assigning care plans to patients. First, the method 100 may extract patient data for patents of interest to the user. The clustering of patients 115 may be performed by using patient data and an existing clustering method such as Agglomerative Hierarchical Clustering (AHC), K-means, DBSCAN, BIRCH, etc. For the current embodiment, AHC will be used. In its simplest form, the clustering algorithm is applied once to form clusters that will be evaluated by the following steps, but another embodiment may allow for the reapplication of the clustering technique on clusters that do not meet the threshold for homogeneity of the composition of the cluster to form smaller (more homogeneous) (sub)clusters. The clustering technique will group together patients that are similar in terms of the input data that characterizes the patients and form distinct clusters that show more differences between clusters than within clusters.

For each cluster a measure of homogeneity is determined 120. The method from copending U.S. Patent Application No. 62/544960, filed Aug. 14, 2017, which is hereby incorporated by reference for all purposes as if fully set forth herein, may be applied, but alternatively also measures such as the Silhouette coefficient, Davies-Bouldin index or Dunn index may be used.

Next, the method 100 determines if any clusters have a homogeneity below a pre-set threshold 125. Any clusters that have a homogeneity below the pre-set threshold are re-clustered 130 (or alternatively they may be discarded) by applying the clustering technique again on the subset of patients in this cluster. The clustering technique used for re-clustering may be the same as that in step 115 or a different technique. Then the homogeneity of the re-clustered clusters is determined 120.

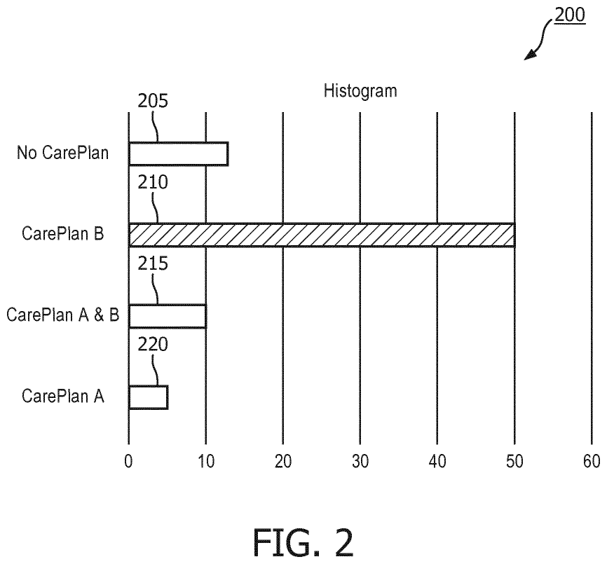

Next, for each patient in the cluster, the care plans/programs that they are enrolled in are retrieved and a histogram is produced 135 to determine the frequency distribution of each combination of care plans/programs within the cluster. It is noted, that this step may be applied to all care plans that the patients are enrolled in, or only certain care plans that relate to specific diseases or concerns. FIG. 2 illustrates a histogram of the care plans showing the frequency distribution of the care plans in which the patients in the cluster are enrolled. This step assumes that there is a set of defined and identifiable care plans/programs that the patients may be enrolled in and which are included in the patient data. In FIG. 2 the histogram includes four categories: no care plan 205; care plan B 210, care plans A and B 215; and care plan A 220 and shows the frequency distribution of the different care plans. The method 100 next determines if the relative frequency of “No Care Plan” is higher than a pre-set threshold (say 40%) 140. If so, this cluster should be discarded from automatic care plan assignment 145 as there is too little evidence that the right care plan will be selected. Also, as this situation may be indicative of bad patient management, an alert may be presented to the user to indicate the possibility of bad patient management.

The method 100 then modifies the care plan assignment for each patient 150 to the most frequent care plan based on the frequency distribution for each patient in this cluster. This could lead to the set the care plan(s) to the preferred option for patients that currently have no care plan assigned. Modifying the care plan 150 may lead to enrolling some patients in an existing care plan, removing some patients off of a care plan, or to switch some patients from one care plan to another care plan. After the care plan assignments are made for each patient, a care giver may approve the assignments. The method 100 may then see to enroll the patients with a change care plans in the new plans and remove them from prior care plans if any.

In another embodiment, a situation may arise where the frequencies of some or all of the most numerous care plans are relatively close to one another so that there is no real statistical difference between them. In this case, the effectiveness of the plans may be used to determine which care plan is assigned to each member of the cluster.

In another embodiment, inclusion/exclusion criteria attached to the care plans may be taken into account assigning a care plan to each patient. For example, if a care plan is specifically designed for diabetes patients, it should only be selected for patients that have diabetes; similarly, a care plan that may exclude patients with certain characteristics should not be assigned to patients with those characteristics.

In further alternative embodiments, the most optimal care plan may be defined through retrospectively analyzing the care plan outcomes linked to a certain patient type (as these end up in the same subgroup of similar patients), thus taking into account other aspects of care plans such as success rates given certain types of patients or financial constraints in order to optimize for these aspects.

In yet another alternative embodiment, a measure of confidence may be derived by comparing the patients whose care plan assignment is suggested to be changed to those that were already on the ‘preferred option’ based upon characteristics that are important to the care plan (e.g., the characteristics used in the inclusion/exclusion criteria). By determining the distance between a (to-be-changed) patient to the group of patients (already on the preferred care plan) and comparing against a threshold(s) (or against other distances observed within the cluster), small distances may be given a high confidence level and those with larger distances a lower level of confidence.

The method 100 may be part of a software tool utilized by the user. The tool may present the modified care plan assignment to the user with the option to make corrections and acknowledge the plan.

Currently, care providers face a technological problem in being able to effectively assign care plans to patients. As the care providers have a large number of a patients with a large of amount of patient data, it is difficult for care providers to identify the best care plans for their patients. The patient care plan assignment method described above solves this problem by taking large amounts of patient data, clustering the patients, determining the most frequently used care plan in each cluster, and the assigning each patient in the cluster to the most frequent care plan.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A computer implemented method for assigning a care plan to a patient, the method comprising:
clustering patients based upon input patient data;
producing a care plan frequency distribution for each cluster based upon the care plans assigned to each patient in the cluster; and
assigning, for each cluster, the most frequent care plan from the frequency distribution for that cluster to each patient in that cluster.

2. The computer implemented method of claim 1, further comprising:
determining the homogeneity of each cluster;
determining if the homogeneity of any cluster is less than a first threshold; and
re-clustering any clusters that have a homogeneity that is less than the first threshold.

3. The computer implemented method of claim 1, further comprising:
determining the homogeneity of each cluster;
determining if the homogeneity of any cluster is less than a first threshold; and
discarding any clusters that have a homogeneity that is less than the first threshold.

4. The computer implemented method of claim 1, further comprising:
determining if the frequency of no care plan of any cluster is greater than a second threshold; and
discarding any cluster with a frequency of no care plan greater than the second threshold.

5. The computer implemented method of claim 1, further comprising:
determining if the frequency of no care plan of any cluster is greater than a second threshold; and
producing an alert to a user indicating the cluster with a frequency of no care plan greater than the second threshold.

6. The computer implemented method of claim 1, further comprising:
producing a confidence measure for each patient whose care plan assignment changed when assigning, for each cluster, the most frequent care plan from the frequency distribution for that cluster to each patient in that cluster.

7. The computer implemented method of claim 6, wherein producing a confidence measure further comprises determining a distance between each patient whose care plan assignment changed and those patients in the cluster that were already assigned to the most frequent care plan from the frequency distribution.

8. The computer implemented method of claim 1, further comprising:
presenting the assigned care plan for a specific patient to a user;
receiving input from the user to modify the care plan assignment of the specific patient based upon patient constraints; and
modifying the care plan assignment of the specific patient based upon the user input.

9. The computer implemented method of claim 1, further comprising:
presenting the assigned care plans for the patients to a user;
receiving input from the user to approve the care plan assignments of the patients; and
initiating enrollment of patients in the assigned care plan for patients whose care plan assignment has changed.

10. A computer implemented method for assigning a care plan to a patient, the method comprising:
clustering patients based upon input patient data;
determining for each cluster which care plan assigned to patients in that cluster provides the best outcome; and
assigning, for each cluster, the care plan that provides the best outcome for that cluster to each patient in that cluster.

11. The computer implemented method of claim 10, wherein the best outcome is one of the best success rate of the care plan or the lowest cost successful care plan.

12. The computer implemented method of claim 10, further comprising:
determining the homogeneity of each cluster;
determining if the homogeneity of any cluster is less than a first threshold; and
re-clustering any clusters that have a homogeneity that is less than the first threshold.

13. The computer implemented method of claim 10, further comprising:
determining the homogeneity of each cluster;
determining if the homogeneity of any cluster is less than a first threshold; and
discarding any clusters that have a homogeneity that is less than the first threshold.

14. The computer implemented method of claim 10, further comprising:
producing a confidence measure for each patient whose care plan changed when assigning, for each cluster, the care plan that provides the best outcome for each cluster to each patient in that cluster.

15. The computer implemented method of claim 14, wherein producing a confidence measure further comprises determining a distance between each patient whose care plan assignment changed and those patients in the cluster that were already assigned to the care plan that provides the best outcome.

16. The computer implemented method of claim 10, further comprising:
presenting the assigned care plan for a specific patient to a user;
receiving input from the user to modify the care plan assignment of the specific patient based upon patient constraints; and
modifying the care plan assignment of the specific patient based upon the user input.

17. The computer implemented method of claim 10, further comprising:
presenting the assigned care plans for the patients to a user;
receiving input from the user to approve the care plan assignments of the patients; and
initiating enrollment of patients in the assigned care plan for patients whose care plan assignment has changed.
